(19) United States
(12) Patent Application Publication
(10) Pub. No.: US 2019/0025269 A1
(43) Pub. Date: Jan. 24, 2019

(54) Thermal ribbon analysis

(71) Applicant: Quantum Signal LLC, Saline, MN (US)

(72) Inventors: David B. Johnson, Saline, MI (US); Robert M. Lupa, Saline, MI (US); Kevin D. Melotti, Ann Arbor, MI (US); Mitchell M. Rohde, Saline, MI (US); Steven M. Rohde, Saline, MI (US); Christopher M. Showers, Royal Oak, MI (US); Michael G. Ploof, Saline, MI (US)

(21) Appl. No.: 16/143,348

(22) Filed: Sep. 26, 2018

Related U.S. Application Data
(63) Continuation of application No. 15/997,999, filed on Jun. 5, 2018.
Application No. 15/997,999 is a continuation of application No. 15/360,125, filed on Nov. 23, 2016, now abandoned.
Application No. 15/360,125 is a continuation of application No. 14/183,239, filed on Feb. 18, 2014, now abandoned.
(60) Provisional application No. 61/766,104, filed on Feb. 18, 2013.

(51) Int. Cl. G01N 33/00; B41J 17/38; B41M 7/00

(52) U.S. Cl. CPC G01N 33/00; B41M 7/009; B41J 17/38

(57) ABSTRACT

Thermal ribbon analysis is achieved via various approaches.

### RELATED APPLICATIONS

The present patent application is a continuation of the non-provisional patent application filed on Jun. 5, 2018, and assigned application Ser. No. 15/997,999, which itself claims priority to the non-provisional patent application filed on Feb. 18, 2014, and assigned application Ser. No. 14/183,239, which itself claims priority to the previously filed provisional patent application of the same title, filed on Feb. 18, 2013, and assigned patent application No. 61/766,104, which is hereby incorporated by reference.

### BACKGROUND

Visual odometry is the process of estimating or measuring the motion of a vehicle through analysis of image sequences collected by a camera mounted on or of the vehicle.

### DETAILED DESCRIPTION

Thermal ribbon analysis is a forensic-focused desktop device that permits fast, efficient, and automated recovery of data on thermal mass transfer or dye diffusion thermal transfer ribbons.

We claim:

1. A thermal ribbon analysis approach.

2. A system and method for thermal ribbon analysis.
